(19) United States
(12) Patent Application Publication
(10) Pub. No.: US 2022/0296752 A1
(43) Pub. Date: Sep. 22, 2022

(54) Full Coverage Disinfectant Spray Apparatus

(71) Applicant: Nathaniel Laster, Roselle, NJ (US)

(72) Inventor: Nathaniel Laster, Roselle, NJ (US)

(21) Appl. No.: 17/204,650

(22) Filed: Mar. 17, 2021

(51) Int. Cl. A61L 2/22

(52) U.S. Cl. CPC A61L 2/22; A61L 2202/25; A61L 2202/16; A61L 2202/15

(57) ABSTRACT

A full coverage disinfectant spray apparatus for disinfecting a room includes a canister having a canister bottom side, a canister sidewall, and a canister top side. The canister contains a pressurized disinfectant sanitizer. A release tube is coupled to the canister. The release tube is coupled to the canister top side. A valve collar is coupled to the release tube. The valve collar selectively releases the pressurized disinfectant sanitizer through the release tube when depressed. A spray head is coupled to the valve collar. The spray head has a head top side, a head bottom side, and a head perimeter. The head bottom side is coupled to the valve collar. A plurality of nozzles is coupled to the spray head. The plurality of nozzles is coupled to the head perimeter and is in fluid communication with the valve collar.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to disinfectant devices and more particularly pertains to a new disinfectant device for disinfecting a room. The device includes a canister body and a spray head. The spray head has four evenly spaced spray nozzles.

### (2) Description of Related Art Including Information Disclosed Under 37 CFR 1.97 and 1.98

The prior art relates to disinfectant devices. Known devices typically include a single spray nozzle oriented upwards. These devices lack a plurality of radially distributed horizontal nozzles to create a wider coverage area.

### BRIEF SUMMARY OF THE INVENTION

An embodiment of the disclosure meets the needs presented above by generally comprising a canister having a canister bottom side, a canister sidewall, and a canister top side. The canister contains a pressurized disinfectant sanitizer. A release tube is coupled to the canister. The release tube is coupled to the canister top side. A valve collar is coupled to the release tube. The valve collar selectively releases the pressurized disinfectant sanitizer through the release tube when depressed. A spray head is coupled to the valve collar. The spray head has a head top side, a head bottom side, and a head perimeter. The head bottom side is coupled to the valve collar. A plurality of nozzles is coupled to the spray head. The plurality of nozzles is coupled to the head perimeter and is in fluid communication with the valve collar.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a full coverage disinfectant spray apparatus according to an embodiment of the disclosure.

FIG. 2 is a front elevation view of an embodiment of the disclosure.

FIG. 3 is an in-use view of an embodiment of the disclosure.

### DETAILED DESCRIPTION OF THE INVENTION

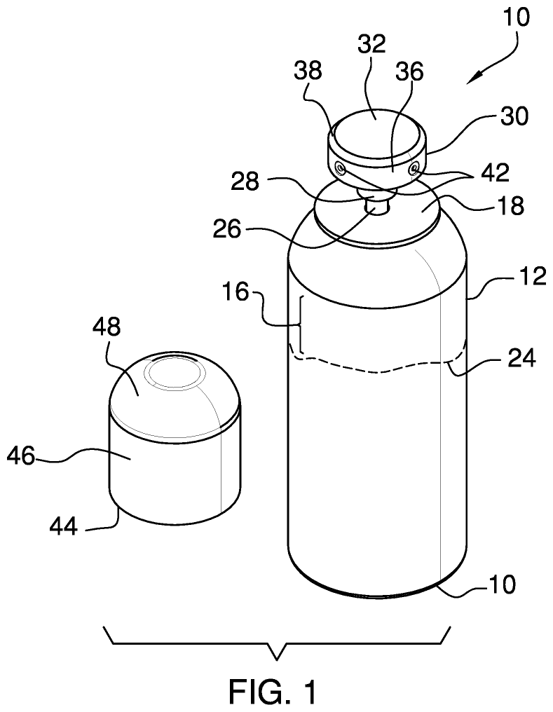

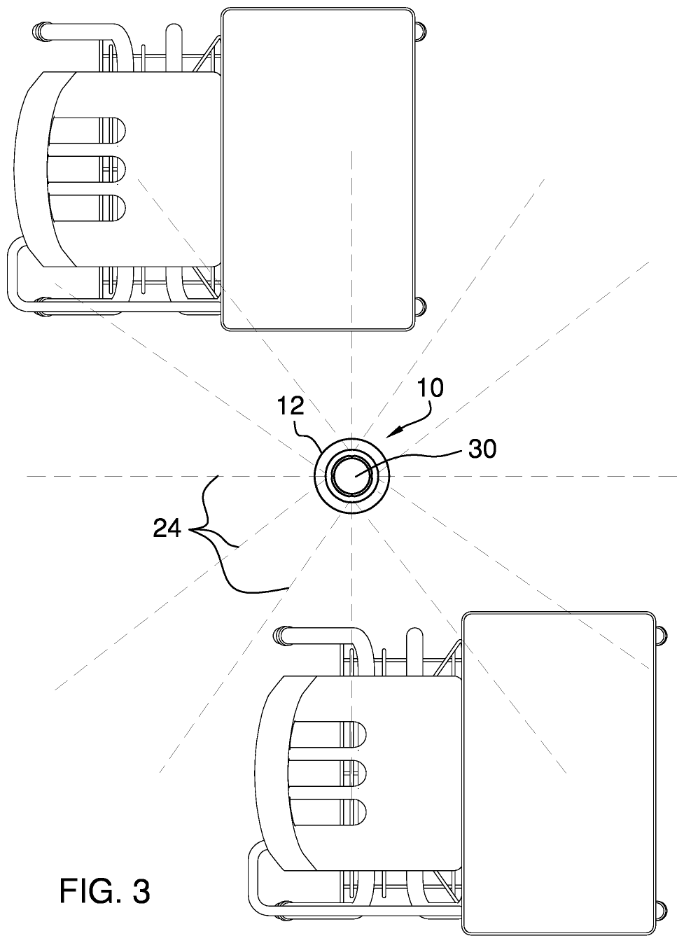

With reference now to the drawings, and in particular to FIGS. 1 through 6 thereof, a new disinfectant device embodying the principles and concepts of an embodiment of the disclosure and generally designated by the reference numeral 10 will be described.

As best illustrated in FIGS. 1 through 6, the full coverage disinfectant spray apparatus 10 generally comprises a canister 12 having a canister bottom side 14, a canister sidewall 16, and a canister top side 18. The canister top side 18 may be circular. The canister sidewall 16 may have a cylindrical portion 20 extending from the cannister bottom side 14 and a filleted portion 22 extending from the cylindrical portion 20 to the cannister top side 18. The canister 12 contains a pressurized disinfectant sanitizer 24.

A release tube 26 is coupled to the canister 12. The release tube 26 is coupled to the canister top side 18. A valve collar 28 is coupled to the release tube 26. The valve collar 28 selectively releases the pressurized disinfectant sanitizer 24 through the release tube 26 when depressed. The valve collar 28 may engage the canister top side 18 such that it can remain depressed without continued contact.

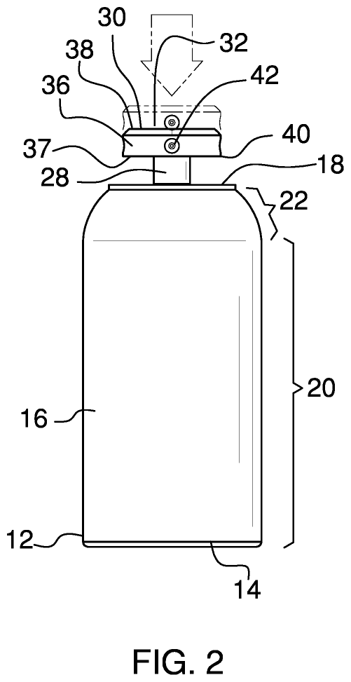

A spray head 30 is coupled to the valve collar 28. The spray head 30 has a head top side 32, a head bottom side 34, and a head perimeter 36. The head bottom side 34 is coupled to the valve collar 28. The head top side 32 and the head bottom side 34 may be circular. The spray head 30 may include a top perimeter 38 and a bottom perimeter 40 of the head top side 32 and the head bottom side 34, respectively. The top perimeter 38 may be chamfered for user comfort. The head perimeter 36 may be concave as shown in FIG. 2.

A plurality of nozzles 42 is coupled to the spray head 30. The plurality of nozzles 42 is coupled to the head perimeter 36 and is in fluid communication with the valve collar 28. The plurality of nozzles 42 is evenly distributed around the head perimeter 36. The plurality of nozzles 42 may be four nozzles 42 each spaced 90° apart. The plurality of nozzles 42 releases the pressurized disinfectant sanitizer 24 as a fine mist that can fill an entire room.

A cap 44 may be selectively engageable with the canister 12 to enclose the spray head 30. The cap 44 may include a cylindrical cap wall 46 and a domed cap top side 48.

In use, the spray head 30 is pressed to depress the valve collar 28. The pressurized disinfectant sanitizer 24 is then released through the plurality of nozzles 42 to fill a room and kill viruses and bacteria. The valve collar 28 and the spray head 30 may delay the release of the pressurized disinfectant sanitizer 24 for up to one minute after being depressed in order to allow the user to leave the room.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A full coverage disinfectant spray apparatus comprising:
a canister having a canister bottom side, a canister sidewall, and a canister top side, the canister containing a pressurized disinfectant sanitizer;
a release tube coupled to the canister, the release tube being coupled to the canister top side;
a valve collar coupled to the release tube, the valve collar selectively releasing the pressurized disinfectant sanitizer through the release tube when depressed;
a spray head coupled to the valve collar, the spray head having a head top side, a head bottom side, and a head perimeter, the head bottom side being coupled to the valve collar; and
a plurality of nozzles coupled to the spray head, the plurality of nozzles being coupled to the head perimeter and being in fluid communication with the valve collar.

2. The full coverage disinfectant spray apparatus of claim 1 further comprising the canister top side being circular, the canister sidewall having a cylindrical portion extending from the cannister bottom side and a filleted portion extending from the cylindrical portion to the cannister top side.

3. The full coverage disinfectant spray apparatus of claim 1 further comprising a cap, the cap being selectively engageable with the canister to enclose the spray head.

4. The full coverage disinfectant spray apparatus of claim 3 further comprising the cap including a cylindrical cap wall and a domed cap top side.

5. The full coverage disinfectant spray apparatus of claim 1 further comprising the head top side and the head bottom side being circular.

6. The full coverage disinfectant spray apparatus of claim 5 further comprising a top perimeter and a bottom perimeter of the head top side and the head bottom side, respectively, the top perimeter being chamfered.

7. The full coverage disinfectant spray apparatus of claim 1 further comprising the head perimeter being concave.

8. The full coverage disinfectant spray apparatus of claim 1 further comprising the plurality of nozzles being evenly distributed around the head perimeter.

9. The full coverage disinfectant spray apparatus of claim 8 further comprising the plurality of nozzles being four nozzles each spaced 90° apart.

10. A full coverage disinfectant spray apparatus comprising:
a canister having a canister bottom side, a canister sidewall, and a canister top side, the canister top side being circular, the canister sidewall having a cylindrical portion extending from the cannister bottom side and a filleted portion extending from the cylindrical portion to the cannister top side, the canister containing a pressurized disinfectant sanitizer;
a release tube coupled to the canister, the release tube being coupled to the canister top side;
a valve collar coupled to the release tube, the valve collar selectively releasing the pressurized disinfectant sanitizer through the release tube when depressed;
a spray head coupled to the valve collar, the spray head having a head top side, a head bottom side, and a head perimeter, the head bottom side being coupled to the valve collar, the head top side and the head bottom side being circular, a top perimeter and a bottom perimeter of the head top side and the head bottom side, respectively, the top perimeter being chamfered, the head perimeter being concave;
a plurality of nozzles coupled to the spray head, the plurality of nozzles being coupled to the head perimeter and being in fluid communication with the valve collar, the plurality of nozzles being evenly distributed around the head perimeter, the plurality of nozzles being four nozzles each spaced 90° apart; and
a cap, the cap being selectively engageable with the canister to enclose the spray head, the cap including a cylindrical cap wall and a domed cap top side.
